(19) United States
(12) Patent Application Publication
(10) Pub. No.: US 2024/0093282 A1
(43) Pub. Date: Mar. 21, 2024

(54) TARGET NUCLEIC ACID DETECTION DEVICE AND MANUFACTURING METHOD FOR SAME

(71) Applicants: Yusuke OSAKI, Kanagawa (JP); Yuuki Yonekawa, Kanagawa (JP); Michie Hashimoto, Tokyo (JP); Hirotaka Unno, Kanagawa (JP)

(72) Inventors: Yusuke OSAKI, Kanagawa (JP); Yuuki Yonekawa, Kanagawa (JP); Michie Hashimoto, Tokyo (JP); Hirotaka Unno, Kanagawa (JP)

(21) Appl. No.: 18/359,706

(22) Filed: Jul. 26, 2023

Related U.S. Application Data
(63) Continuation of application No. PCT/JP2022/003307, filed on Jan. 28, 2022.

(30) Foreign Application Priority Data
Jan. 28, 2021 (JP) 2021-012417

(51) Int. Cl. C12Q 1/6851; B01L 3/00; B01L 7/00; C12Q 1/70

(52) U.S. Cl. CPC C12Q 1/6851; B01L 3/502707; B01L 3/502715; B01L 7/52; C12Q 1/701; B01L 2200/12; B01L 2200/16; B01L 2300/18; C12Q 2600/166

(57) ABSTRACT

The present invention has an object to develop and provide a target nucleic acid detection device having a low copy number of IPC nucleic acids placed therein, and a method for producing the device, to avoid a decrease caused in the detection sensitivity to a target nucleic acid through competitive inhibition of the target nucleic acid by addition of an internal positive control (IPC) nucleic acid. Provided is a target nucleic acid detection device in which a low copy number of IPC nucleic acids that do not inhibit the amplification reaction of a target nucleic acid is preliminarily placed in a reaction well.

### CROSS-REFERENCE TO RELATED APPLICATIONS

This application is a U.S. bypass continuation of International Application No. PCT/JP2022/003307, filed on Jan. 28, 2022, and which claims the benefit of priority to Japanese Application No. 2021-012417, filed on Jan. 28, 2021. The content of each of these applications is hereby incorporated by reference in its entirety.

### REFERENCE TO A SEQUENCE LISTING

The present application is accompanied by an XML file as a computer readable form containing the sequence listing entitled, “005604USBP-SL.xml”, created on Jul. 24, 2023, with a file size of 3,142 bytes, the content of which is hereby incorporated by reference in its entirety.

### TECHNICAL FIELD

The present invention relates to a target nucleic acid detection device containing a low copy number of internal positive controls, and to a method for producing the device.

### BACKGROUND ART

In recent years, a higher sensitivity achieved in analysis technology has allowed a measuring object to be measured in a unit of the number of molecules. For example, a nucleic acid analysis technology in which amplifying a target nucleic acid present in trace amounts in a test specimen is followed by minutely analyzing the amplification product obtained is widely utilized in various fields such as food inspection, environmental inspection, medical care, and criminal investigation. The nucleic acid analysis technology often involves verifying that an analyte specimen contains no measuring object, i.e., is negative, and accordingly requires an extremely high detection sensitivity and an evaluation of the result of the detection.

Typical examples of the nucleic acid analysis technology include real-time polymerase chain reaction (PCR). “Real-time PCR” is a PCR process in which the fluorescence corresponding to the amount of nucleic acid amplified is detected in a timely manner, and a nucleic acid of interest in the analyte is quantitated. In a quantitative analysis of a nucleic acid specimen using the real-time PCR or the like, it is important in an evaluation of the result that a nucleic acid specimen is compared with a calibration curve prepared on the basis of a result in which a series of nucleic acid specimens including various in copy numbers or molecules are measured. However, the evaluation of the result is based on the premise that a quantitative test has been established. For the evaluation, a positive control (herein often referred to as a “PC”) and an internal positive control (herein often referred to as an “IPC”) are usually used.

The PC works as a control for evaluating an amplification reagent and reaction conditions such as temperature in PCR. That a PC reaction is normal means that the PCR reaction conditions have no problem, and that the measurement has been made normally.

On the other hand, in the nucleic acid analysis technology, a test nucleic acid specimen is often a trace of nucleic acid prepared from a test specimen such as soil, plant, skin, or feces, and such a test nucleic acid specimen often contains a substance that inhibits the nucleic acid amplification reaction, such as polyphenol, humic acid, fluvic acid, tannin, or melanin that is derived from the test specimen. An IPC is multiply amplified together with a test nucleic acid specimen in the same reaction liquid, and works as a control for verifying the inhibitory influence that the above-described inhibitory substance has on PCR. That the amplification of an IPC is verified in a reaction container means that the influence of the inhibitory substance has not been present or has been slight. Additionally, when the amplification of a target nucleic acid is not enabled to be verified simultaneously, the evaluation can be that no target nucleic acid of interest is present in the test nucleic acid specimen. However, it is known that, with a reaction liquid supplemented with an IPC, the detection sensitivity is decreased, compared with a reaction liquid that generally amplifies a target nucleic acid alone (Non-patent Document 1). This is because, in a multiplex amplification reaction in which a plurality of objects are amplified simultaneously in the same reaction liquid, the amplification reaction of an IPC and the amplification reaction of a target nucleic acid compete with each other.

Among quantitative analysis methods, a quantitative method by competitive PCR that conversely makes use of this competitive inhibition of the multiplex amplification reaction is known (Non-patent Documents 2 and 3).

On the other hand, it has been made clear that, to avoid competitive inhibition in a multiplex amplification reaction, the concentration of IPCs can be made low.

However, in cases where IPCs are added in low copy numbers, the copy number per container tends to vary between the production lots, and it is difficult to keep the accuracy of each container constant. Because of this, various technologies for producing a device containing a low copy number of nucleic acids are studied (Patent Documents 1 to 3).

The intensity of competition in the competitive inhibition in a nucleic acid amplification reaction varies depending on the relative amount of template nucleic acid. In view of this, the present inventors have produced a target nucleic acid detection device in which a low copy number of IPC nucleic acids that do not inhibit the amplification reaction of a target nucleic acid is preliminarily placed in a reaction well. In addition, to decrease unevenness caused in IPC nucleic acids between specimens by dispensing the IPC nucleic acids in low copy numbers, the present inventors have come to solve the above-mentioned problems by dispensing a predetermined number of cells containing a predetermined copy number of nucleic acids of interest such as IPC nucleic acids.

Hitherto, an invention made using a low copy number of IPCs is known. For example, an Internal Positive Control (315-08241) from Nippon Gene Co., Ltd. is commercially available as an existing product. This product is a low copy number of IPCs the concentration of which is preliminarily adjusted to 20 (17 to 23) copies/μL by digital PCR. However, this product is intended to be used, for example, for evaluation of the inhibitory influence when a genetic test is performed, and whether the product can be used to avoid a decrease caused in the detection sensitivity to a target nucleic acid by multiplex amplification is not disclosed. In addition, there is a problem in that the copy number of IPCs per 1 μL is 17 to 23 copies, i.e., the range of variation is too large.

Patent Document 4 discloses an invention that relates to an internal control nucleic acid molecule to be used for a nucleic acid amplification system. This patent discloses pseudo-randomly producing all of a forward primer binding site, a reverse primer binding site, and an amplifiable region for the purpose of developing the following: an internal control molecule designed as part of a comprehensive internal control system that can be introduced false-negatively; and a method for using the internal control molecule. However, the patent does not disclose a solution regarding the inhibition caused to the amplification of a target nucleic acid by addition of an IPC and the resulting decrease in detection sensitivity.

### SUMMARY OF THE INVENTION

### Technical Problem

The present invention has an object to develop and provide: a target nucleic acid detection device that can avoid a decrease caused in the detection sensitivity to a target nucleic acid through competitive inhibition by addition of an IPC nucleic acid; and a method for producing the device.

### Solution to Problem

The present invention provides the following.

(1) A target nucleic acid detection device including a base material and a nucleic acid receiving part, the nucleic acid receiving part containing a predetermined copy number of internal positive control (IPC) nucleic acids either on a surface of or in an internal space of or both on the surface of and in the internal space of the nucleic acid receiving part, the IPC nucleic acid having a specific base sequence, and the predetermined copy number being a copy number according to which a ratio of the copy number of the IPC nucleic acids to an estimated copy number of target nucleic acids is 200 or less.

(2) A method for producing a target nucleic acid detection device, including: an IPC dispensing step of dispensing a predetermined number of cells into one or more nucleic acid receiving parts on a base material, the cell containing a predetermined copy number of IPC nucleic acids having a specific base sequence; and a nucleic acid extracting step of extracting a nucleic acid from the cell.

The present specification encompasses the disclosure of Japanese Patent Application No. 2021-012417 that serves as a basis for the priority of the present application.

### Effects of Invention

A target nucleic acid detection device according to the present invention enables the inhibitory influence of an inhibitory substance on a nucleic acid amplification reaction to be evaluated with addition of a low copy number of IPC nucleic acids, and can decrease the influence of competitive inhibition due to the IPC, and inhibit a decrease in the detection sensitivity to a target nucleic acid.

### BRIEF DESCRIPTION OF THE DRAWINGS

FIG. 1 is a graph illustrating the results of Example 2; the ordinate represents the Ct value, and the abscissa represents the copy number of IPCs added; in this graph, the EGFR gene and 600G were used as a target nucleic acid and an IPC nucleic acid respectively; to draw a logarithmic graph, 0 as the copy number of the IPC nucleic acids on the abscissa is represented by 1, on which basis calculations are made: the Ct values are plotted for each of the copy numbers of the target nucleic acids:and the square plot (▪), rhombus plot (♦), triangle plot (▴), and circle plot (●) represent the Ct values for 100 copies, 500 copies, 1000 copies, and 5000 copies respectively:

FIG. 2 is a graph illustrating the variations in the Ct values obtained in Example 2; the ordinate represents the ratio (%) of the Ct value for each of the copy numbers of the IPC nucleic acids to the Ct value given when the copy number of the IPC nucleic acids was 0 (the abscissa value=1) in FIG. 1, i.e., when the EGFR gene alone as a target nucleic acid was added; the abscissa represents the ratio of the copy number of the IPC nucleic acids to the copy number of the target nucleic acids; to draw a logarithmic graph, 0 as the copy number of the IPC nucleic acids on the abscissa is represented by 0.001, on which basis calculations are made; the ratios between the Ct values are plotted for each of the copy numbers of the target nucleic acids; the square plot (▪), rhombus plot (♦), triangle plot (▴), and circle plot (●) represent the ratios for 100 copies, 500 copies, 1000 copies, and 5000 copies respectively; and as verified, the Ct value for any copy number of the EGFR genes was decreased as the copy number of the IPC nucleic acids was increased;

FIG. 3 is a graph illustrating a decrease in the plateau position obtained in Example 3; the ordinate represents the Rn value after 50 cycles; the “Rn value” represents the intensity of a fluorescent signal: the Rn value is a value normalized by dividing the fluorescence emission intensity of a reporter pigment of a probe to be used for qPCR by the fluorescence emission intensity of a passive reference pigment: the abscissa represents the ratio of the copy number of the IPCs to the copy number of the target nucleic acids (IPC nucleic acids/target nucleic acids); to draw a logarithmic graph, 0 as the copy number of the IPC nucleic acids is represented by 0.001, on which basis calculations are made: the Rn values are plotted for each of the copy numbers of the target nucleic acids; and the square plot (▪), rhombus plot (♦), triangle plot (▴), and circle plot (●) represent the Rn values for 100 copies, 500 copies, 1000 copies, and 5000 copies respectively;

FIG. 4 is a graph illustrating the results of Example 4; this is an electrophoretic image illustrating how an amplification product obtained by a PCR reaction under the same conditions as in Example 3 was electrophoresed with a 4% Agarose gel; and at both ends, a size marker (Low DNA Mass Ladder, manufactured by Thermo Fisher Scientific Inc.) is given:

FIG. 5 is a graph illustrating the variations in the Ct values obtained in Example 5; the ordinate represents the ratio (%) of the Ct value for each of the copy numbers of the IPC nucleic acids to the Ct value given when the copy number of the IPC nucleic acids was 0, i.e., when the novel coronavirus N2 sequence alone as a target nucleic acid was added; the abscissa represents the ratio of the copy number of the IPC nucleic acids to the copy number of the target nucleic acids:as in FIG. 2, to draw a logarithmic graph, 0 as the copy number of the IPC nucleic acids on the abscissa is represented by 0.001, on which basis calculations are made; the ratios between the Ct values are plotted for each of the copy numbers of the target nucleic acids; the square plot (▪), rhombus plot (♦), triangle plot (▴), circle plot (●) and cross plot (x) represent the ratios for 5 copies, 50 copies, 500 copies, 5000 copies and 50000 copies of target nucleic acids respectively; and as verified, the Ct value of the N2 sequence was increased as the copy number of the IPC nucleic acids was increased; and

FIG. 6 is a graph representing a calibration curve in accordance with which the copy number of the N2 sequences was estimated from the Ct value of the N2 sequence: the ordinate represents the Ct value of the N2 sequence, and the abscissa represents the copy number of the N2 sequences; and this graph illustrates a calibration curve of each of the copy numbers of IPC nucleic acids (600G) added.

### MODE FOR CARRYING OUT THE INVENTION

### 1. Target Nucleic Acid Detection Device

### 1-1. Overview

A first aspect of the present invention is a target nucleic acid detection device. The device according to the present invention is characterized by including a nucleic acid receiving part containing a low copy number of internal positive control (IPC) nucleic acids.

A target nucleic acid detection device according to the present invention enables the influence of an inhibitory substance on a nucleic acid amplification reaction to be verified with addition of an IPC nucleic acid, and can inhibit a decrease caused in the detection sensitivity to a target nucleic acid by inhibition due to competition with the IPC nucleic acid.

### 1-2. Definition of Terms

The main terms used herein are defined below.

(1) Nucleic Acid

As used herein, a “nucleic acid” refers to a biopolymer the constituent unit of which is a nucleotide in principle, and in which nucleotides are linked via a phosphodiester bond. The nucleic acid usually refers to a naturally occurring nucleic acid such as a DNA (encompassing a cDNA), an RNA, or a combination thereof, in which naturally occurring nucleotides alone are linked, but herein can encompass a non-naturally occurring nucleic acid the whole or part of which is composed of non-naturally occurring nucleotides or the like. In the present invention, a naturally occurring nucleic acid or a non-naturally occurring nucleic acid can be selected suitably in accordance with the purpose.

As used herein, a “naturally occurring nucleotide” refers to a nucleotide existing in nature. Specifically, the naturally occurring nucleotide includes a deoxyribonucleotide that constitutes a DNA, and has any base of adenine, guanine, cytosine, and thymine, or a ribonucleotide that constitutes an RNA, and has any base of adenine, guanine, cytosine, and uracil.

As used herein, a “non-naturally occurring nucleotide” refers to a nucleotide that does not exist in nature, and is constructed artificially, or chemically modified artificially. In general, the non-naturally occurring nucleotide includes a nucleotide containing anon-naturally occurring nucleoside, base analogue, or modified base having a property and/or structure similar to the property and/or structure of the above-described naturally occurring nucleoside or base, in addition to a nucleotide-like substance having a property and/or structure similar to the property and/or structure of the above-described naturally occurring nucleotide. Examples of the “non-naturally occurring nucleoside” include an abasic nucleoside, arabinonucleoside, 2′-deoxyuridine, α-deoxyribonucleoside, β-L-deoxyribonucleoside, and other sugar-modified nucleosides. Examples of the “base analogue” include a 2-oxo(1H)-pyridine-3-yl group, 5-position-substituted-2-oxo(1H)-pyridine-3-yl group, 2-amino-6-(2-thiazolyl)purine-9-yl group, 2-amino-6-(2-thiazolyl)purine-9-yl group, and 2-amino-6-(2-oxazolyl)purine-9-yl group. Examples of the “modified base” include: modified pyrimidines such as 5-hydroxy cytosine, 5-fluorouracil, and 4-thiouracil; modified purines such as 6-methyladenine and 6-thioguanosine; and other heterocycle bases.

As used herein, a “non-naturally occurring nucleic acid” is an artificially constructed nucleic acid analogue having a structure and/or property similar to the structure and/or property of a naturally occurring nucleic acid. Examples include a bridged nucleic acid (BNA/LNA: Bridged Nucleic Acid/Locked Nucleic Acid), peptide nucleic acid (PNA: Peptide Nucleic Acid), peptide nucleic acid having a phosphate group (PHONA), and morpholino nucleic acid. Examples may also include chemically modified nucleic acids and nucleic acid analogues such as methyl phosphonate DNA/RNA, phosphorothioate DNA/RNA, phosphoramidate DNA/RNA, and 2′-O-methyl DNA/RNA.

The phosphate group, sugar, and/or base in a nucleic acid herein may be labeled with a labeling substance, if desired. The position of labeling for the labeling substance can be determined suitably in accordance with the characteristics of the labeling substance and the purpose of the use, and is usually preferably, but not limited to, the 5′ end and/or 3′ end. As a labeling substance, any substance known in the art can be utilized. Examples include a radioisotope, fluorescent substance, quencher, chemiluminescent substance, DIG, biotin, and magnetic beads. The “radioisotope” refers to an element that emits radial rays among isotopes having different mass numbers. Examples include ³²P, ³³P, and ³⁵S.

The “fluorescent substance” is a substance having a property of absorbing an excited light having a specific wavelength to come into an excited state, and emitting fluorescence % when coming back into the original ground state. Examples include, FITC, Texas, Texas Red (registered trademark), cy3, cy5, cy7, FAM, HEX, VIC (registered trademark), JOE, ROX, TET, Bodipy 493, NBD, TAMRA, Quasar (registered trademark) 670, Quasar (registered trademark) 705, CAL Fluor (registered trademark) Red 610, SYBR Green (registered trademark), Eva Green (registered trademark), SYTOX Green (registered trademark), fluorescamine or derivatives thereof, fluorescein or derivatives thereof, azo substances, or rhodamine or derivatives thereof, coumarin or derivatives thereof, pyrene or derivatives thereof, and cyanine or derivatives thereof. These may be used singly or in combination of two or more kinds thereof.

The “quencher” refers to a substance having a property of absorbing the excitation energy of the fluorescent substance to inhibit the fluorescence. Examples include AMRA, DABCYL, BHQ-1, BHQ-2, and BHQ-3. The “chemiluminescent substance” refers to a substance having a property of being excited by a chemical reaction, and then emitting a difference in energy as light when brought back into the ground state. Examples include an acridinium ester.

Herein, the shape of a nucleic acid is not limited. The nucleic acid can take any suitable shape, as desired. For example, the nucleic acid may be a linear nucleic acid such as an oligo nucleotide, or a cyclic nucleic acid such as a plasmid.

(2) Internal Positive Control Nucleic Acid (IPC Nucleic Acid)

As used herein, an “internal positive control nucleic acid” (herein often referred to as an “IPC nucleic acid) refers to a template nucleic acid used as an internal positive control (IPC) in a target nucleic acid detection device according to the present invention. As above-described, an IPC is a control for evaluating the inhibitory influence of the above-described inhibitory substance on PCR by multiply amplifying the IPC together with a test nucleic acid specimen in the same reaction liquid. As above-described, that the amplification of an IPC nucleic acid is verified in a reaction container means that the influence of the inhibitory substance has not been present or has been slight. Additionally, when the amplification of a target nucleic acid is not enabled to be verified simultaneously, the evaluation can be that the test nucleic acid specimen contains no target nucleic acid of interest, i.e., is negative, as long as the extraction efficiency of the test nucleic acid specimen used is not low.

(3) Positive Control Nucleic Acid (PC Nucleic Acid)

As used herein, a “positive control nucleic acid” (herein often referred to as a “PC nucleic acid) refers to a template nucleic acid used as a positive control (PC) in a target nucleic acid detection device according to the present invention. In principle, the whole or part of the positive control nucleic acid has the same base sequence as the target nucleic acid.

As above-described, the PC is a control for evaluating an amplification reagent and reaction conditions such as temperature in PCR. That a PC reaction is normal means that the PCR reaction conditions have no problem, and that the measurement has been made normally. If a PC reaction is normal when a multiplex amplification reaction is performed with an IPC nucleic acid, the evaluation can be, in addition to the above-mentioned effect, that the amplification of a target nucleic acid is not inhibited even in the presence of an IPC.

(4) Reference Nucleic Acid Specimen

As used herein, a “reference nucleic acid specimen” refers to a reference material composed of a nucleic acid. A “reference material” (RM) is a standard material for determining a measurement value in the measurement of a chemical substance, and contains a predetermined amount of nucleic acid. A nucleic acid as a reference nucleic acid specimen herein is a DNA, unless otherwise specified, and the nucleic acid in the present invention is a nucleic acid containing the same base sequence as a target nucleic acid.

(5) Target Nucleic Acid

As used herein, a “target nucleic acid” refers to a nucleic acid of interest as an object that is to be measured or detected, and can be contained in a test nucleic acid specimen, the target nucleic acid is usually a naturally occurring nucleic acid such as a DNA or an RNA. The kind of the target nucleic acid is not limited as long as the target nucleic acid can be amplified. For example, the target nucleic acid may be the base sequence of a specific gene or part thereof, or may be a specific region on a genomic DNA. The base sequence of the target nucleic acid may be a coding region, or may be a non-coding region such as a spacer or an intron. In addition, the base sequence can be not only an endogenous base sequence but also an exogenous base sequence, and furthermore, can be not only a natural base sequence but also an artificial base sequence constructed by a genetic recombination technology, genome-editing, or the like.

The base sequence length of the target nucleic acid is not particularly limited, and is preferably a length that can be amplified by a nucleic acid amplification reaction such as PCR. For example, the base sequence length can be 5 bases or more, 8 bases or more, 10 bases or more, 12 bases or more, 15 bases or more, 18 bases or more, 20 bases or more, 23 bases or more, 25 bases or more, 28 bases or more, 30 bases or more, 35 bases or more, 40 bases or more, 50 bases or more, 60 bases or more, 70 bases or more, 80 bases or more, 90 bases or more, or 100 bases or more, and 20 K (20000) bases or less, 18 K bases or less, 15 K bases or less, 12 K bases or less, 10 K bases or less, 8.0 K bases or less, 5.0 bases or less, 3.0 K bases or less, 2.0 K bases or less, 1.8 K bases or less, 1.5 bases or less, 1.2 K bases or less, 1.0 K bases or less, 800 bases or less, 600 bases or less, 500 bases or less, 400 bases or less, 300 bases or less, or 200 bases or less.

In the present invention, the copy number of target nucleic acids contained in a test nucleic acid specimen is preferably lower. This is because an IPC may not be needed in the detection of a target nucleic acid in some of the cases where the copy number of target nucleic acids is high.

(6) Test Nucleic Acid Specimen

As used herein, a “test nucleic acid specimen” refers to a nucleic acid specimen that can be contained in a test specimen, and in addition, can contain the above-described target nucleic acid. The test nucleic acid specimen is directly used as a nucleic acid specimen in a target nucleic acid detection device according to the present invention. Usually, all the nucleic acids contained in a test specimen are test objects, and thus, are regardless of the kind, such as a DNA (encompassing a genomic DNA, plasmid DNA, and the like) or an RNA (encompassing an mRNA, tRNA, rRNA, snRNA, miRNA, and the like). The test nucleic acid specimen is a naturally occurring nucleic acid contained in a test specimen, but may contain a non-naturally occurring nucleic acid.

(7) Test Specimen

As used herein, a “test specimen” refers to a specimen to be used as a test object in which a nucleic acid is to be analyzed. The test specimen is not limited to any kind, and can be any specimen that can contain a test nucleic acid specimen. For example, the test specimen is part (a nucleus, cell, tissue, or organ) of an organism or a product derived from the organism, or a soil, the air, and water (encompassing lake water, seawater, river water, sewage and the like) containing the part of an organism or the derivation product derived from the organism, or the like. For example, specific examples of the part of an organism include a nail, hair, skin tissue, mucosa tissue bodily fluid (encompassing, for example, blood, lymph, tissue liquid, spinal fluid, semen, and vaginal fluid) and the like, and specific examples of the product derived from the organism include feces and urine, vomit and excreta, and organism-derived liquid (encompassing, for example, urine, saliva, sputum, nasal discharge, tear, sweat, breast milk, pleural effusion, ascitic fluid, and peritoneal washing, and the like).

(8) Copy

As used herein, a “copy” refers to a replica of a basic unit when using a specific nucleic acid such as the above-described IPC nucleic acid, PC nucleic acid, target nucleic acid, or reference nucleic acid specimen as the basic unit.

As used herein, a “copy number” refers to the number of basic units when using the above-described specific nucleic acid as the basic unit, and the copy number is the total number of template nucleic acids and nucleic acid replicas. Usually, a sense strand is used as the basic unit, but in the case of a duplex nucleic acid, any one of a sense strand and an antisense strand can be a template in a nucleic acid amplification reaction, and thus, the duplex nucleic acid counts as 2 copies.

As used herein, an “estimated copy number” refers to an estimated value of the copy number of target nucleic acids that can be contained in a test specimen or a test nucleic acid specimen. The estimated value is estimated from past measurement values under environmental conditions, such as the state, period of collection, and place of collection of a test specimen or a test nucleic acid specimen, and similar conditions thereto, and thus, is regardless of accuracy. A target nucleic acid detection device according to the present invention allows the detection of a low copy number of target sequences in a test specimen or a test nucleic acid specimen, and thus, the estimated copy number may be low. The estimated copy number can be, for example, but is not limited to, 1 copy or more, 2 copies or more, 4 copies or more, 8 copies or more, 10 copies or more, 15 copies or more, 20 copies or more, 30 copies or more, 40 copies or more, or 50 copies or more, and 20000 copies or less, 18000 copies or less, 15000 copies or less, 12000 copies or less, 10000 copies or less, 8000 copies or less, 5000 copies or less, 3000 copies or less, 2000 copies or less, 1500 copies or less, 1000 copies or less, 900 copies or less, 800 copies or less, 700 copies or less, 600 copies or less, 500 copies or less, 400 copies or less, or 300 copies or less.

(9) Multiplex Amplification

As used herein, “multiplex amplification” refers to amplification performed simultaneously by a plurality of kinds of nucleic acid fragments to be amplified in one reaction liquid in a nucleic acid amplification reaction such as PCR. A “multiplex amplification reaction” refers to a reaction that causes multiplex amplification. Herein, multiplex amplification by PCR is referred to as “multiplex PCR”.

As above-mentioned, a multiplex amplification reaction involves mutual competition for a reaction reagent and a primer among the respective amplification reactions in a reaction liquid, and thus, tends to give a lower detection sensitivity to an object intended to be amplified than a single amplification reaction.

### 1-3. Constitution

A target nucleic acid detection device according to the present invention includes a base material and a nucleic acid receiving part as fundamental constituents. The constitution of each constituent will be specifically described below.

1-3-1. Base Material

A “base material” is a support that is itself rigid, and affords a given shape to the target nucleic acid detection device.

The material of the base material is not particularly limited, and can be selected suitably in accordance with the purpose, as long as the material has no influence on a nucleic acid, does not inhibit the detection reaction of the nucleic acid, and has rigidity enough to retain a given shape. The material may be either an organic material or an inorganic material. Examples of the organic material include a synthetic resin, natural resin, silicone material, cellulose structure (wood, paper, and the like), natural fiber (silk, fur, cotton, spongin fiber, and the like) and the like. Examples of the inorganic material include glass (encompassing glass fiber and quartz), earthenware (ceramic, enamel, and the like), semiconductor, metal, mineral, carbon fiber, and calcium phosphate structure (bone, tooth, seashell, and the like) and the like.

The material preferably has not only properties of being easily processed into a desired shape, not rotten, durable, and light but also a high thermal conductivity, considering that a target nucleic acid detection device according to the present invention is used for a nucleic acid amplification reaction. For example, without limitation, the material is suitably a synthetic resin. Examples of the synthetic resin include polypropylene (PP), polyethylene terephthalate (PET), polystyrene (PS), polycarbonate (PC), TAC (triacetyl cellulose), polyimide (PI), nylon (Ny), low-density polyethylene (LDPE), medium-density polyethylene (MDPE), vinyl chloride, vinylidene chloride, polyphenylene sulfide, polyethersulfone, polyethylene naphthalate, urethane acrylate and the like. Examples of the silicone material include polydimethyl siloxane (PDMS) and the like.

In addition, the base material that constitutes a target nucleic acid detection device according to the present invention may be not only a single material but also a combination of a plurality of kinds of materials. For example, the material for the below-mentioned nucleic acid receiving part can be different from the material for another base material. In a specific example, it is possible that the material for the body of the base material is made of metal, and the nucleic acid receiving part alone is made of polypropylene. Alternatively, the base material can have a layer structure composed of a plurality of different materials. For example, it is possible that the surface of the base material is formed of a thin layer of polypropylene, under which a polystyrene layer for giving rigidity to the device is disposed as an underlayer.

The shape of the base material is not particularly limited, and can be selected suitably in accordance with the purpose. Examples include a plate, chip, slide, dish, connected tube or the like.

1-3-2. Nucleic Acid Receiving Part

(1) Nucleic Acid Receiving Part

The “nucleic acid receiving part” is a part that receives an IPC nucleic acid and/or a test nucleic acid specimen to serve as a place for a nucleic acid amplification reaction in a target nucleic acid detection device according to the present invention.

The nucleic acid receiving part is disposed on the surface and/or in the inside of the base material in a target nucleic acid detection device according to the present invention.

As long as the shape of the nucleic acid receiving part allows the retention of a nucleic acid and the performance of a nucleic acid amplification reaction, the shape is not particularly limited, can be selected suitably in accordance with the purpose, and can be, for example, a recessed shape having a flat bottom, round bottom, U-shaped bottom, or V-shaped bottom, or a planar shape having sections. Specific examples of the recessed shape include, but are not limited to, a well.

The receiving capacity of the nucleic acid receiving part is also not limited. The receiving capacity can be selected suitably in accordance with the purpose. Considering the volume of a specimen to be used in a common nucleic acid detection reaction, the receiving capacity is preferably 1 μL to 2000 μL, 3 μL to 1500 μL, 5 μL to 1200 μL, or 10 μL to 1000 μL.

The material of the nucleic acid receiving part is not particularly limited, and can be selected suitably in accordance with the purpose, as long as the material has no influence on a nucleic acid, and does not inhibit the detection reaction of the nucleic acid. As with the material of the base material, any one of an organic material and an inorganic material can be selected. The material of the nucleic acid receiving part may be the same as or different from the material of the base material.

The color of the nucleic acid receiving part is not particularly limited. For example, the nucleic acid receiving part can be transparent, translucent, colored, or completely light-blocking, and may have any color.

One or more nucleic acid receiving parts can be disposed per target nucleic acid detection device. In cases where real-time PCR or digital PCR that evaluates a target nucleic acid quantitatively is used, a plurality of nucleic acid receiving parts are preferably included in the target nucleic acid detection device. In this case, the number of nucleic acid receiving parts per target nucleic acid detection device can be, for example, but is not limited to, 2, 4, 8, 16, 32, 64, 96, 128, 192, or 384. These can be achieved with connected microtubes, a multi-well plate, or the like.

(2) Lidding Means

The “lidding means” is a lid configured to hermetically seal the nucleic acid receiving part to prevent contamination and the outflow of a reaction liquid, and is an optional constituent of the nucleic acid receiving part. The form of the lidding means is, for example, but not limited to, a cap form that conforms to the inner wall diameter of the nucleic acid receiving part, or a film form that covers the opening of the nucleic acid receiving part. The cap form may be integrated with the target nucleic acid detection device, or may a separable form that is removable. In cases where the target nucleic acid detection device includes a plurality of nucleic acid receiving parts, at least one of the nucleic acid receiving parts can be configured to be hermetically sealed.

(3) IPC Nucleic Acid

The nucleic acid receiving part contains a predetermined copy number of IPC nucleic acids.

The IPC nucleic acid is composed of a DNA, unless otherwise specified. The DNA of the IPC nucleic acid is composed of a naturally occurring nucleotide, in principle, but may partially contain a non-naturally occurring nucleotide that can work as a template in a nucleic acid amplification reaction.

The base sequence of the IPC nucleic acid is not particularly limited as long as the base sequence of a region to be amplified (an amplification region) does not form a higher-order structure owing to a change in the temperature during a nucleic acid amplification reaction. In addition, the base sequence of the IPC nucleic acid may be a base sequence derived from part of a naturally occurring nucleic acid, such as a specific gene or a specific region on a genomic DNA, but is preferably an artificially designed base sequence the whole or part of which does not exist in nature. In light of performing multiplex amplification, the base sequence of the IPC nucleic acid has an identity of 60% or less, 55% or less, 50% or less, 45% or less, 40% or less, 35% or less, or 30% or less to the base sequence of an amplification region of the target nucleic acid or the PC nucleic acid.

The base sequence length of the IPC nucleic acid is not particularly limited, and the amplification base length is preferably within +30%, within ±25%, within +20%, within +15%, or within or 10% of the amplification base length of a target nucleic acid in the same reaction liquid. For example, in cases where the amplification base length of a target nucleic acid in the same reaction liquid is 100 bases, the amplification base length of the IPC nucleic acid is preferably 70 to 130 bases, 75 to 125 bases, 80 to 120 bases, 85 to 115 bases, or 90 to 110 bases.

The “predetermined copy number” is the preliminarily set copy number of nucleic acid molecules (for example, IPC nucleic acids or PC nucleic acids), and involves having a given or higher degree of accuracy after dispensing. This predetermined copy number in the present application is characterized by having higher accuracy and higher reliability in number than a copy number obtained by conventional serial dilution, dispensing with a pipette, or the like (calculated estimated value). In particular, even a region having a low copy number at less than 1000 has a controlled value that has a small dispersion and is not achieved by the Poisson distribution. For the controlled value, a coefficient of variation, CV, that represents uncertainty is preferably a value that generally satisfies CV<1/√x and CV<20%, with respect to an average copy number x. For example, it is preferable that the IPC nucleic acids contained in each nucleic acid receiving part are less than 1000 copies, and that the CV value of the copy number is less than 20%. Here, 1/√x represents a dispersion in the Poisson distribution, assuming λ=x. Here, k is one of the parameters in the Poisson distribution. A target nucleic acid detection device containing such a predetermined copy number can be produced by a production method according to the below-mentioned third aspect.

In a target nucleic acid detection device according to the present invention, the predetermined copy number of the IPC nucleic acids contained per nucleic acid receiving part can be suitably adjusted with the estimated copy number of target nucleic acids subjected to multiplex amplification together with the IPC nucleic acids in the same nucleic acid receiving part. Specifically, the ratio of the copy number of IPC nucleic acids to the estimated copy number of target nucleic acid specimens (IPC nucleic acids/target nucleic acids) can be, for example, 0.0001 or more, 0.009 or more, 0.008 or more, 0.007 or more, 0.006 or more, 0.005 or more, 0.004 or more, 0.003 or more, 0.002 or more, 0.001 or more, 0.09 or more, 0.08 or more, 0.07 or more, 0.06 or more, 0.05 or more, 0.04 or more, 0.03 or more, 0.02 or more, or 0.01 or more, and 200 or less, 180 or less, 160 or less, 140 or less, 120 or less, 100 or less, 80 or less, 60 or less, 40 or less, 20 or less, 10 or less, 9 or less, 8 or less, 7 or less, 6 or less, 5 or less, 4 or less, 3 or less, 2 or less, 1 or less, 0.9 or less, 0.8 or less, 0.7 or less, 0.6 or less, 0.5 or less, 0.4 or less, 0.3 or less, 0.2 or less, or 0.1 or less. A detecting object of a target nucleic acid detection device according to the present invention is a test nucleic acid specimen in which the copy number of target nucleic acids is estimated to be low, and thus, the copy number of IPC nucleic acids can be 1 copy or more and 10000 copies or less, 1 copy or more and 5000 copies or less, 1 copy or more and 3000 copies or less, 1 copy or more and 2000 copies or less, 1 copy or more and 1000 copies or less, 1 copy or more and 800 copies or less, 1 copy or more and 600 copies or less, 1 copy or more and 500 copies or less, 1 copy or more and 400 copies or less, 1 copy or more and 30) copies or less, 1 copy or more and 200 copies or less, 1 copy or more and 100 copies or less, 1 copy or more and 90 copies or less, 1 copy or more and 80 copies or less, 1 copy or more and 70 copies or less, 1 copy or more and 60 copies or less, 1 copy or more and 50 copies or less, 1 copy or more and 40 copies or less, 1 copy or more and 20 copies or less, or 1 copy or more and 10 copies or less.

In cases where the target nucleic acid detection device contains a plurality of nucleic acid receiving parts, it is preferable that IPC nucleic acids are contained in all the nucleic acid receiving parts. This is because the conditions for nucleic acid amplification reactions in all the nucleic acid receiving parts can be adjusted with the IPC. However, the target nucleic acid detection device may include one or more nucleic acid receiving parts containing no IPC nucleic acid. In cases where a plurality of nucleic acid receiving parts contain IPC nucleic acids, the copy number of the IPC nucleic acids may be the same or different between the nucleic acid receiving parts, and considering the purpose of the addition of an IPC nucleic acid, the copy number is preferably the same, in principle.

(4) PC Nucleic Acid

In cases where the target nucleic acid detection device contains a plurality of nucleic acid receiving parts, at least one of the nucleic acid receiving parts may contain a PC nucleic acid in place of a target nucleic acid specimen. To the nucleic acid receiving part containing a PC nucleic acid, no test nucleic acid specimen is added, in principle.

The PC nucleic acid is composed of a DNA, unless otherwise specified. The DNA of the PC nucleic acid is composed of a naturally occurring nucleotide, in principle, but may partially contain a non-naturally occurring nucleotide that can work as a template in a nucleic acid amplification reaction.

In principle, the base sequence of the PC nucleic acid is the same as that of a target nucleic acid, or has the same consecutive base sequences as that of a target nucleic acid. Accordingly, the base sequence length of the PC nucleic acid can be the same or approximately the same as a target sequence. In addition, the PC nucleic acid preferably has the same amplification region as the target nucleic acid, and in this case, amplification is performed using the same primer as the target nucleic acid amplification primer.

The PC nucleic acid may be preliminarily placed in the nucleic acid receiving part together with the IPC nucleic acid, or alternatively, can be added during a PCR reaction. However, in cases where the copy number of the PC nucleic acids contained per nucleic acid receiving part is based on a predetermined copy number in the same manner as with the IPC nucleic acid, the PC nucleic acids are placed in the nucleic acid receiving part preliminarily.

The PC nucleic acids can be placed together with IPC nucleic acids in a plurality of nucleic acid receiving parts. In this case, the predetermined copy number of the PC nucleic acids placed in each nucleic acid receiving part may be the same between the parts, but the PC nucleic acids are preferably contained at a plurality of different levels. In an example of such a case, 1 copy, 2 copies, 4 copies, 8 copies, 16 copies, 32 copies, 64 copies, and 96 copies per nucleic acid receiving part are dispensed. When this is done, the IPC nucleic acids placed simultaneously in each nucleic acid receiving part preferably have the same copy number between the parts. In the presence of IPC nucleic acids, the limit value (lower detection limit value) at which the target sequence can be amplified can be known, and in addition, a calibration curve for a target nucleic acid can be prepared on the basis of the amount of amplification product of the PC nucleic acids contained at each level. Thanks to this, using a target nucleic acid amplification primer in the presence of IPC nucleic acids to perform a nucleic acid amplification reaction on a detection nucleic acid specimen allows the quantitation of a target sequence contained in the detection nucleic acid specimen.

In cases where the target nucleic acid detection device contains a nucleic acid receiving part containing no IPC nucleic acid, a PC nucleic acid alone can be placed in the nucleic acid receiving part. Thanks to this, the influence that competitive inhibition has on PC nucleic acids owing to the presence of an IPC nucleic acid can be evaluated. In cases where a plurality of nucleic acid receiving parts having PC nucleic acids alone placed therein are provided, the predetermined copy number of the PC nucleic acids placed in each nucleic acid receiving part can be on a different level. When this is done, the copy number of the PC nucleic acids can be the same as the predetermined copy number of the PC nucleic acids placed together with the IPC nucleic acids.

In a target nucleic acid detection device according to the present invention, the IPC nucleic acids and/or PC nucleic acids in a nucleic acid receiving part may be suspended in a solution such as a buffer to form a liquid, or may be in the form of a dry solid. The nucleic acids in such a dry state can decrease the possibility that the nucleic acids are decomposed by an enzyme, besides can be stored at normal temperature, and thus, are preferable.

### 1-4. Effect

A target nucleic acid detection device according to the present invention enables the influence of an inhibitory substance in a nucleic acid amplification reaction to be evaluated using a low copy number of IPCs, and simultaneously can inhibit a decrease in the detection sensitivity to a target nucleic acid, in which the decrease can be caused by competitive inhibition in a multiplex amplification reaction in which IPC nucleic acids are added.

### 2. Target Nucleic Acid Detection Kit

### 2-1. Overview

A second aspect of the present invention is a target nucleic acid detection kit.

With a target nucleic acid detection kit according to the present invention, a competitive PCR inhibiting effect due to multiplex amplification with IPC can be reduced minimally while the inhibition of PCR by an inhibitory substance is checked with IPCs, even in cases where target nucleic acids that can be contained in a test specimen are in low copy numbers. This enables high-sensitivity and accurate results to be obtained.

### 2-2. Constitution

A target nucleic acid detection kit according to the present invention includes a target nucleic acid detection device and an IPC amplification primer as fundamental constituents. In addition, the kit includes, as an optional constituent, any of various reaction reagents for PCR, besides a target nucleic acid amplification primer or a PC amplification primer. Each constituent will be specifically described below.

2-2-1. Target Nucleic Acid Detection Device

The “target nucleic acid detection device” is a target nucleic acid detection device described in the first aspect. The specific constitution of the device has already been described in the first aspect, and thus, the specific description is omitted here.

2-2-2. IPC Amplification Primer

An “IPC amplification primer” in the present aspect is a primer that can specifically amplify a predetermined region of an IPC nucleic acid contained in a nucleic acid receiving part of the target nucleic acid detection device. The IPC amplification primer is composed of a forward primer (herein referred to as a “Fw primer”) and a reverse primer (herein referred to as a “Rv primer”). Each primer is composed of a naturally occurring nucleotide and/or a non-naturally occurring nucleotide. The primer is usually composed of a naturally occurring nucleotide of a DNA or an RNA, and is preferably a DNA that has high stability, is easy to synthesize, and is low-cost. If desired, part of the primer can be combined with a non-naturally occurring nucleotide such as an LNA/BNA.

A base sequence and base length of the Fw primer and the Rv primer in the IPC amplification primer are not particularly limited as long as the primers are designed to be capable of specifically amplifying the amplification region of an IPC nucleic acid. Usually, the base sequence and base length of each primer are designed so that the amplification region can be sandwiched between both primers. An IPC is used assuming multiplex amplification, and thus, as the base sequence of the IPC amplification primer, the base sequence of a region that at least is not annealed to a target nucleic acid, and has high specificity is desirably selected. Without limitation, the base sequence and base length are preferably designed so that the Tm value can be in the range of 55° C. or more and 80° C. or less, preferably 60° C. or more and 75° C. or less, and so that the base length and the base sequence can allow hybridization with a base sequence of consecutive 18 bases or more and 35 bases or less, 19 bases or more and 34 bases or less, 20 bases or more and 33 bases or less, 21 bases or more and 32 bases or less, 22 bases or more and 31 bases or less, or 23 bases or more and 30 bases or less in the amplification region of the sequence.

The target nucleic acid detection kit may contain two or more pairs of primers. Examples of cases where a plurality of primer pairs are desired include cases where an amplification region of an IPC nucleic acid is amplified with a nested primer and cases where the kit contains a target nucleic acid amplification primer or a PC amplification primer.

2-2-3. Target Nucleic Acid Amplification Primer/PC Amplification Primer

In the present aspect, a “target nucleic acid amplification primer” is a primer that enables a predetermined region of a target nucleic acid to be specifically amplified using a target nucleic acid detection device. A “PC amplification primer” is a primer that enables a predetermined region of a PC nucleic acid contained in the nucleic acid receiving part to be specifically amplified. These are optional constituents of a target nucleic acid detection kit according to the present invention.

As above-mentioned, a PC nucleic acid herein has the same base sequence as a target nucleic acid, in principle, and thus, the PC amplification primer is the same as a target nucleic acid amplification primer, in principle.

The target nucleic acid amplification primer has a basic constitution in accordance with the IPC amplification primer except that an object to be amplified is a target nucleic acid. However, in a target nucleic acid detection kit according to the present invention, any target nucleic acid is amplified and detected. Accordingly, the kind of a target nucleic acid amplification primer and the base sequence constituting the primer differ, depending on the kind of a target nucleic acid to be detected. Accordingly, a target nucleic acid detection kit containing a target nucleic acid amplification primer can be understood to be a detection kit dedicated to a target nucleic acid that is a target for the target nucleic acid amplification primer.

2-2-4. Others

Besides, the target nucleic acid detection kit may contain, as an optional constituent, a nucleic acid amplification reagent, a labeling reagent, a kit protocol, or the like, if desired.

Examples of the nucleic acid amplification reagent include, nucleic acid polymerase, dNTP (dGTP, dCTP, dATP, dTTP, or dUTP), Mg²⁺, a buffer such as Tris-HCl keeping an optimal pH (pH 7.5 or more and pH 9.5 or less), and nuclease-free water.

The labeling reagent is not particularly limited as long as the reagent as a label can distinguish the amplification of a base sequence to be detected, and the reagent can be selected suitably in accordance with the purpose. A reagent that can subject a disperse phase to optical labeling such as fluorescent labeling or luminous labeling is preferable. The reagent that performs optical labeling is not particularly limited, and can be selected suitably in accordance with the purpose. For example, a commercially available labeling reagent, for example, a nucleic acid probe such as TaqMan (registered trademark) probe or Molecular Beacon or an intercalator such as SYBR Green (registered trademark) or Eva Green, can be utilized.

### 3. Method for Producing Target Nucleic Acid Detection Device

### 3-1. Overview

A third aspect of the present invention is a method for producing a target nucleic acid detection device.

The production method according to the present invention allows the production of a target nucleic acid detection device according to the first aspect.

### 3-2. Method

A method for producing a target nucleic acid detection device according to the present invention includes an IPC dispensing step and a nucleic acid extracting step as fundamental steps. The method can also include a nucleic acid introducing step, a nucleic acid labeling step, and a PC dispensing step as optional steps. Each step will be described below.

3-2-1. Nucleic Acid Introducing Step

The “nucleic acid introducing step” is a step of introducing a predetermined copy number of nucleic acids of interest into a cell. The present step is an optional step that precedes the nucleic acid labeling step, the IPC dispensing step, and the PC dispensing step.

In the present invention, the “nucleic acid of interest” refers to an IPC nucleic acid or a PC nucleic acid to be dispensed via a cell that is a carrier.

A cell into which a nucleic acid of interest is to be introduced is not particularly limited, and any cell can be used. The cell can be selected suitably in accordance with the purpose. Specific examples of the cell include eukaryotic cells, prokaryotic cells, multicellular biological cells, and unicellular biological cells.

Examples of the eukaryotic cell include animal cells, insect cells, plant cells, fungi, seaweeds, and protozoans. Without limitation, an animal cell or a fungus is suitable as a cell in the present production method. The animal cell may be any of a primary cultured cell collected directly from a tissue or an organ, a passage cell obtained by subculturing the primary cultured cell for several generations, and a cell line, and can be selected suitably in accordance with the purpose. The cell may be any of a differentiated cell and an undifferentiated cell.

The differentiated cell is not particularly limited, and can be selected suitably in accordance with the purpose. Examples of the differentiated cell include: epidermal cells such as a stellate cell, Kupffer cell, vascular endothelial cell, sinusoidal endothelial cell, fibroblast, osteoblast, osteoclast, periodontium-derived cell, and epidermal keratinized cell; a tracheal epithelial cell: a gastrointestinal epithelial cell: a uterocervical epithelial cell; a lactocyte: a pericyte; a smooth muscle myocyte; a nephrocyte: a pancreas islet; neurocytes such as a peripheral neurocyte and an optic nerve cell: a chondrocyte; an osteocyte: a hepatocyte that is a parenchymal cell of a liver: endothelial cells such as a corneal endothelial cell; epithelial cells such as a corneal epithelial cell; and myocytes such as a cardiomyocyte.

The undifferentiated cell is not particularly limited, and can be selected suitably in accordance with the purpose, and examples of the undifferentiated cell include; pluripotent stem cells such as a mesenchymal stem cell having pluripotency; unipotent stem cells such as an endothelial precursor cell having unipotency: embryonic stem cells; and iPS cells.

The fungus is not particularly limited, and can be selected suitably in accordance with the purpose. Examples of the fungus include filamentous fungi and yeasts. Among these, a yeast is preferable as a cell to be used in the production method according to the present invention in terms of allowing the regulation of the cell cycle and the use of a haploid. The kind and mutant of the yeast are not particularly limited, and a budding yeast, a fission yeast, or the like can be selected suitably in accordance with the purpose. For example, as a budding yeast, a pheromone (sex hormone)-sensitive Bar-1-deficient variant that allows the control of the cell cycle to the G1 phase is suitable as a cell in the present invention. A Bar-1-deficient variant allows a decrease in the abundance ratio of another yeast strain that does not allow the control of the cell cycle, and thus, can prevent an increase in a predetermined copy number in a cell received in the nucleic acid receiving part.

Examples of the prokaryotic cell include eubacteria and archaebacteria. The prokaryotic cell can be selected suitably in accordance with the purpose.

In the production method according to the present invention, the cell may be used singly or in combination of two or more kinds thereof.

For all the above-described cells, a change caused in the amount of the nucleic acid in the cell by cell division can be decreased utilizing a dead cell, besides a mutant that allows the regulation of the cell cycle, such as a Bar-1-deficient variant yeast. A dead cell allows the prevention of a change caused in the amount of nucleic acid in the cell by a cell division in a process after the preparation of a nucleic acid specimen.

3-2-2. Nucleic Acid Labeling Step

The “nucleic acid labeling step” is a step of labeling a nucleic acid in a cell. The present step is a pre-treatment step to be performed so that cells can be dispensed on a number basis in the subsequent dispensing steps (the IPC dispensing step and the PC dispensing step).

To label a nucleic acid, a known labeling method or staining method can be used. To label a nucleic acid, a phosphate group, sugar, base, and/or double helix can be labeled with a labeling substance. The position of labeling can be determined suitably in accordance with the characteristics of the labeling substance and the purpose of the use. Examples of the labeling substance include a radioisotope, fluorescent substance, and chemiluminescent substance.

The “radioisotope” refers to an element that emits radial rays among isotopes having different mass numbers. Examples include ³²P, ³³P, and ³⁵S.

The “fluorescent substance” is a substance having a property of absorbing an excited light having a specific wavelength to come into an excited state, and emitting fluorescence when coming back into the original ground state. Known examples include, but are not limited to, fluorescent pigments, intercalators, and fluorescent proteins.

Examples of the “fluorescent pigment” include FITC, Texas, Texas Red (registered trademark), Alexa Fluor 405, Alexa Fluor 488, Alexa Fluor 647, Alexa Fluor 700, Pacific Blue, DyLight 405, DyLight 550, DyLight 650, PE-Cy5 (phycoerythrin-cyanin 5), PE-Cy7 (phycoerythrin-cyanin 7), PE (phycoerythrin), PerCP (peridinin chlorphyll protein), PerCP-Cy5.5 (peridinin chlorphyll protein-cyanin 5.5), APC (Allophycocyanin), Hoechst 33258, Hoechst 33342, Hoechst 34580, cy3, cy5, cy7, FAM, HEX, VIC (registered trademark), JOE, ROX, TET, Bodipy 493, NBD, TAMRA, Quasar (registered trademark) 670, Quasar (registered trademark) 705, CAL Fluor (registered trademark) Red 610, SYBR Green (registered trademark), Eva Green (registered trademark), SYTOX Green (registered trademark), fluorescamine or derivatives thereof, fluorescein or derivatives thereof, azos, or rhodamine or derivatives thereof, coumarin or derivatives thereof, pyrene or derivatives thereof, and cyanine or derivatives thereof.

The “intercalator” refers to a low-molecular-weight compound to be inserted in parallel between a pair of bases in the double helix structure of a DNA. Examples include, but are not limited to, ethidium bromide (EB), propidium iodide (PI), acridine orange (AO), and DAPI (4′,6-diamidino-2-phenylindole).

Examples of the “fluorescent protein” include EGFP, CFP, YFP, RFP and the like.

The “chemiluminescent substance” refers to a substance having a property of being excited by a chemical reaction, and then emitting a difference in energy as light when brought back into the ground state. Examples include an acridinium ester.

To label a nucleic acid, the above-described labeling substance may be used singly or in combination of two or more kinds thereof.

3-2-3. IPC Dispensing Step

The “IPC dispensing step” is a step of dispensing a predetermined number of cells into one or more nucleic acid receiving parts on a base material. The cells to be dispensed contain a predetermined copy number of IPC nucleic acids having a specific base sequence.

As used herein, a “predetermined number” refers to the preliminarily set number of the cells, and has a given or higher degree of accuracy owing to dispensing the cells. These cells contain a predetermined copy number of IPC nucleic acids per cell, and thus, the present step is, in other words, a step of dispensing a predetermined copy number of IPC nucleic acids into nucleic acid receiving parts by dispensing a predetermined number of cells into the nucleic acid receiving parts.

In the present step, a predetermined number of cells containing a predetermined copy number of IPC nucleic acids are dispensed. Without limitation, the cells can be dispensed in the form of a liquid, i.e., a cell suspension. When this is done, the predetermined copy number of IPC nucleic acids dispensed per nucleic acid receiving part can be, but is not limited to, 1 copy or more and 10000 copies or less, 1 copy or more and 5000 copies or less, 1 copy or more and 3000 copies or less, 1 copy or more and 2000 copies or less, 1 copy or more and 1000 copies or less, 1 copy or more and 800 copies or less, 1 copy or more and 600 copies or less, 1 copy or more and 500 copies or less, 1 copy or more and 400 copies or less, 1 copy or more and 300 copies or less, 1 copy or more and 200 copies or less, 1 copy or more and 100 copies or less, 1 copy or more and 90 copies or less, 1 copy or more and 80 copies or less, 1 copy or more and 70 copies or less, 1 copy or more and 60 copies or less, 1 copy or more and 50 copies or less, 1 copy or more and 40 copies or less, 1 copy or more and 20 copies or less, or 1 copy or more and 10 copies or less. The cells can be dispensed with the number of the cells controlled to achieve 10000 copies or less. The volume of the cell suspension can be 1 fL to 1 μL, 100 fL to 0.5 μL, 500 fL to 100 nL, or 1 nL to 50 nL.

In cases where the cells are dispensed into a plurality of the nucleic acid receiving parts in the production method according to the present invention, the cells are preferably dispensed with the number of the cells controlled in such a manner that the copy number of the IPC nucleic acids contained in each nucleic acid receiving part is the same between the parts.

Any known dispensing method can be used as a method for dispensing a predetermined number of cells containing a predetermined copy number of IPC nucleic acids into the nucleic acid receiving parts of the target nucleic acid detection device. Examples include a flow cytometry method and a method using a discharge mechanism.

The flow cytometry method allows thousands to millions of cells labeled with a fluorescent substance or the like to be quantitatively measured one by one with a sheath fluid in a short time. Each cell undergoes correlational analysis and statistical analysis with a plurality of pieces of measurement information, and on the basis of the information, the cells can be fractionated. Specifically, for example, it is possible to measure the labeling amount or labeling intensity (for example, the fluorescent amount or fluorescent brightness) of each cell, and fractionate the cells containing a predetermined amount of DNAs (for example, haploids or diploids) on the basis of the measurement information.

Even in cases where the target nucleic acid detection device has a plurality of nucleic acid receiving parts such as wells, the flow cytometry method allows any number of cells to be dispensed into each well for placement.

On the other hand, examples of the method using a discharge mechanism include, but are not limited to, a fluid transport passage technique, on-demand technique, continuous technique and the like. In the method using the mechanism, a label contained in a cell is detected when the cells are dispensed. The label can be detected, for example, with a detector included in the discharge mechanism. The detector is not particularly limited, and can be selected suitably in accordance with the purpose. For example, an optical detection method can be used.

The fluid transport passage technique is a method in which a predetermined number of cells are discharged in the form of microdroplets into a nucleic acid receiving part from a nozzle through a passage that allows the transportation of a fluid (liquid) containing the cells.

Examples of the on-demand technique include a discharge head technique and the like. Typical examples of the discharge head technique include an inkjet technique and the like. Examples of the discharge head in the inkjet technique include a discharge head of an electrostatic type, thermal type, pressurizing type or the like. The type is preferably, but not limited to, a pressurizing type.

Examples of the pressurizing type include a type in which the liquid is pressurized using a piezoelectric element, a type in which the liquid is pressurized using a valve such as a solenoid valve and the like. The pressurizing type is advantageous in that no electrode is attached, and that no concern is caused about burning and sticking to a heater portion, compared with a thermal type.

Dispensing a predetermined copy number of IPC nucleic acids in the state of being contained in a cell allows regulating, on the basis of the number of cells, the copy number of IPC nucleic acids to a predetermined copy number in a nucleic acid receiving part, and producing an IPC nucleic acid specimen having a small error between specimens.

3-2-4. Nucleic Acid Extracting Step

The “nucleic acid extracting step” is a step of extracting a nucleic acid from a cell. As a method for extracting a nucleic acid from a cell, a method known in the art can be used. For example, a method described in Sambrook, J. et. al., (1989) Molecular Cloning: a Laboratory Manual Second Ed., Cold Spring Harbor Laboratory Press, Cold Spring Harbor, New York can be referred to. Besides, various kits that allow the preparation of various nucleic acids such as RNAs and genomic DNAs are commercially available from manufacturers associated with life science, and can be utilized.

3-2-5. PC Dispensing Step

The “PC dispensing step” is a step in which, in cases where the cells are dispensed into a plurality of nucleic acid receiving parts in the IPC dispensing step, a predetermined number of cells containing a predetermined copy number of PC nucleic acids are dispensed into any one or more of the nucleic acid receiving parts. That is, the present step is an optional step that is performed, if desired, in cases where a plurality of nucleic acid receiving parts exist in a target nucleic acid detection device to be produced.

The basic constitution of the present step is in accordance with the constitution of the IPC dispensing step except that an object to be dispensed is a PC nucleic acid. In the IPC dispensing step, IPC nucleic acids are dispensed into nucleic acid receiving parts in such a manner that the copy number of IPC nucleic acids is the same between the nucleic acid receiving parts, in principle, but, in cases where PC nucleic acids are dispensed into a plurality of nucleic acid receiving parts in the present step, the copy number of PC nucleic acids may be the same or different between the nucleic acid receiving parts. To prepare a calibration curve for quantitation of target nucleic acids, the PC nucleic acids are preferably dispensed in such a manner that the copy number of the PC nucleic acids is different between the nucleic acid receiving parts. If desired, the PC nucleic acids may be dispensed into a nucleic acid receiving part containing no IPC nucleic acid.

The copy number of the PC nucleic acids to be dispensed is not limited, and, the PC nucleic acids are preferably dispensed in such a manner that the ratio of the copy number of the IPC nucleic acids to the copy number of the PC nucleic acids (IPC nucleic acids/PC nucleic acids) is 200 or less per nucleic acid receiving part.

The present step can be performed before, after, or simultaneously with the IPC dispensing step.

### EXAMPLES

Specific aspects and embodiments of the present invention will be described with reference to the following Examples, and the present invention is not limited to the following Examples.

### Example 1

(Purpose)

In Example 1, IPC nucleic acids were prepared and dispensed into nucleic acid receiving parts, and values for evaluating the dispensing accuracy were allocated.

(Method)

1. Preparation of IPC Nucleic Acid-Containing Cells

(1) Preparation of Genetically Engineered Yeast

As a carrier cell having 1 copy of an IPC nucleic acid, a budding yeast YIL015W BY4741 (ATCC4001408) was used. Using a DNA 600-G (NMIJ CRM 6205-a from National Institute of Advanced Industrial Science and Technology) as an IPC nucleic acid, 1 copy of a plasmid containing a selection marker URA3 in tandem with the DNA 600-G was introduced, by homologous recombination, into the BAR1 region on the genomic DNA of the budding yeast to prepare a genetically engineered yeast having 1 copy of an IPC nucleic acid.

(2) Culture and Synchronization of Cell Cycle

The genetically engineered yeast cultured in a 50 g/L YPD culture medium (CLN-630409, manufactured by Takara Bio Inc.) was dispensed in an amount of 90 mL into an Erlenmeyer flask, and 900 μL of an al-Mating Factor acetate salt (T6901-5MG, manufactured by Sigma-Aldrich, hereinafter referred to as an α-factor) prepared to be 500 μg/mL using a Dulbecco's phosphate buffered saline solution (DPBS) (14190-144, manufactured by Thermo Fisher Scientific Inc.) was added. Using a BioShaker (BR-23FH, manufactured by Taitec Corporation), the resulting solution was incubated at a shaking rate of 250 rpm at a temperature of 28° C. for 2 hours to obtain a yeast suspension in which the yeast was synchronized to the G0/G1 phase.

(3) Immobilization of Yeast

To a centrifuge tube (VIO-50R, manufactured by As One Corporation), 45 mL of a yeast suspension the cell cycle synchronization of which was verified was transferred, and centrifuged at a rotational speed of 3000 rpm for 5 minutes using a centrifugal separator (F16RN, manufactured by Hitachi, Ltd.), and the supernatant was removed to obtain yeast pellets. To the yeast pellets obtained, 4 mL of formalin (062-01661, manufactured by Fujifilm Wako Pure Chemical Corporation) was added, left to stand for 5 minutes, and then centrifuged to remove the supernatant, and 10 mL of ethanol was added to suspend the solution, whereby an immobilized yeast suspension was obtained.

(4) Nucleic Acid Staining

The immobilized yeast suspension was separated in an amount of 200 μL, washed with DPBS once, and then re-suspended in 480 μL of DPBS. 20 μL of 20 mg/mL RNaseA (318-06391, manufactured by Nippon Gene Co., Ltd.) was added, and then incubated at 37° C. for 2 hours using the BioShaker. Then, 25 μL of 20 mg/mL proteinase K (TKR-9034, manufactured by Takara Bio Inc.) was added, and incubated at 50° C. for 2 hours using Petite Cool (Petite Cool MiniT-C, manufactured by Wakenbtech Co. Ltd.). Finally, 6 μL of 5 mM SYTOX Green Nucleic Acid Stain (S7020, manufactured by Thermo Fisher Scientific Inc.) was added, and the nucleic acids in the cell were stained for 30 minutes in the shade.

(5) Dispersion

The yeast suspension stained was dispersed at an output of 30% for 10 seconds using an ultrasonic homogenizer (LUH150, manufactured by Yamato Scientific Co., Ltd.) to obtain a yeast suspension ink.

2. Dispensing of IPC Nucleic Acid-containing Cells

(1) Dispensing and Cell Measurement

Using the yeast suspension ink prepared, the number of yeasts in a droplet was counted by the below-described method, during which a target nucleic acid detection device having nucleic acid receiving parts containing a known number of cells was produced. Specifically, a droplet forming device was used to discharge the yeast suspension ink at 10 Hz sequentially into each well of a 96 plate (MicroAmp 96-well Reaction plate, manufactured by Thermo Fisher Scientific Inc.), using a piezoelectric discharge head (manufactured by Ricoh Company, Ltd.) as a droplet discharging means.

The ink was photographed using a high-sensitivity camera (sCMOS pco.edge, manufactured by Tokyo Instruments. Inc.) as a light-receiving means for the yeasts in the droplet discharged. Using a YAG laser (Explorer ONE-532-200-KE, manufactured by Spectra-Physics, Inc.) as a light source, and using an image processing software Image J as a particle counting means for the image photographed, the image was processed to count the number of cells, and a plate of known cell number, which contains one cell per well, was produced.

(2) Extraction of Nucleic Acid

Using Tris-EDTA (TE) Buffer and ColE1 DNA (312-00434, manufactured by Fujifilm Wako Pure Chemical Corporation), ColE1/TE was prepared in such a manner that the ColE1 DNA was 5 ng/μL, and then, in the resulting mixture, Zymolyase (registered trademark) 100T (07665-55, manufactured by Nacalai Tesque, Inc.) was dissolved to prepare a 1 mg/mL Zymolyase solution.

To each well of the plate of known cell number, 4 μL of Zymolyase solution was added, and the resulting mixture was incubated at 37.2° C. for 30 minutes to dissolve the cell wall (nucleic acid extraction), and then heat-treated at 95° C. for 2 minutes to produce a reference device.

Next, to consider the reliability of the result obtained from the plate of known cell number, a plate in which the known cell number was one was produced to calculate uncertainty in one cell number. For each predetermined copy number, the uncertainty was calculated in various copy numbers by the method described in (3) below.

(3) Calculation of Uncertainty

In the present Example, calculations were made using the number of cells in a droplet, the copy number of nucleic acids amplifiable in the cell, the number of cells in a well, and contamination as factors of uncertainty caused in the number of cells and the copy number of IPC nucleic acids per well by dispensing.

The number each used as the number of cells in a droplet was as follows: a number obtained by analyzing an image of a droplet discharged from the discharging means, and counting the number of cells in the droplet; and a number obtained by allowing each droplet discharged by the discharging means to land on a glass slide, and using a microscope to observe the number of cells in the droplet allowed to land.

The copy number of nucleic acids (cell cycle) in a cell was calculated using the ratio (99.5%) of the cells falling under the G1 phase of the cell cycle, and the ratio (0.5%) of the cells falling under the G2 phase.

The number of cells in a well was obtained by counting the number of droplets discharged and allowed to land in the well. As a result, all the droplets in the 96 samples were allowed to land in the wells, and thus, the factor of the number of cells in the well was excluded from the calculation of an uncertainty.

Contamination was checked by three trials, subjecting 4 μL of a filtrate of the ink to real-time PCR to see whether the ink liquid was contaminated with any amplifiable nucleic acid other than the reagent in the cell. As a result, all of the results obtained in three trials were the lower detection limit, and thus, a factor of contamination was also excluded from the calculation of an uncertainty.

The uncertainty was determined as follows. A standard deviation was determined from the measurement value for each factor. Next, a standard uncertainty was determined by multiplying the standard deviation by a coefficient of sensitivity to equalize with the unit of the amount of measurement. Then, a combined standard uncertainty was determined by squaring each of standard uncertainties and summing them. Since the combined standard uncertainty covered the values alone in the range of approximately 68% of the normal distribution, an uncertainty could be obtained as an expanded uncertainty by doubling the combined standard uncertainty considering the range of approximately 95% of the normal distribution. The result is illustrated in the budget sheet of Table 1. Here, the uncertainties for the number of cells in the well and the contamination are excluded from the Table.

TABLE 1

| | | | | | | | Standard |
|---|---|---|---|---|---|---|---|
| | | | | | | Coefficient | Uncertainty |
| | Factor of | Value | Probability | | Standard | of | (Unit of Amount |
| Symbol | Uncertainty | (±) | Distribution | Divisor | Uncertainty | Sensitivity | of Measurement) |
| u1 | Number of Cells | 0.1037 | | 1 | 0.1037 | 1.0290 | 0.1067 |
| | in Droplet | | | | | copies/cell | copy |
| u2 | Copy Number | 0.0709 | | 1 | 0.0709 | — | 0.0709 |
| | of Nucleic Acids | copy | | | copy | | copy |
| | in Cell (Cell Cycle) | | | | | | |
| uc | Combined Standard | | Normal | | | | 0.1281 |
| | Uncertainty | | Distribution | | | | copy |
| U | Expanded | | Normal | | | | 0.2562 |
| | Uncertainty | | Distribution | | | | copy |
| | | | (k = 2) | | | | |

The “symbol” in the Table denotes a given symbol made to correspond to a factor of uncertainty.

In the Table, the “value (+)” is an experimental standard deviation of the average value, and obtained by dividing the calculated experimental standard deviation by a value of the square root of the number of data.

In the Table, the “probability distribution” is a probability distribution that a factor of uncertainty has. For type A evaluation of uncertainty, a blank was left, and for type B evaluation of uncertainty, any one of normal distribution or rectangular distribution was written. In the present Example, the type A evaluation of uncertainty alone was performed, and thus, the spaces for u1 and u2 under the probability distribution are blank.

In the Table, the “divisor” means a figure that normalizes an uncertainty obtained from each factor. In the Table, the “standard uncertainty” is a value obtained by dividing a “value (+)” by a “divisor”.

In the Table, the “coefficient of sensitivity” denotes a value used for equalization to the unit of the amount of measurement.

Subsequently, the average predetermined copy number and uncertainty of the nucleic acid specimens contained in the wells were calculated. The results are tabulated in Table 2. The coefficient of variation, a CV value, was calculated by dividing a value of uncertainty by an average predetermined copy number.

TABLE 2

| | | Coefficient |
|---|---|---|
| Predetermined | | of Variation, |
| Copy Number | | CV Value |
| Average | Uncertainty | (%) |
| 1.02E+00 | 1.28E−01 | 12.60 |
| 2.03E+00 | 1.81E−01 | 8.91 |
| 4.07E+00 | 2.56E−01 | 6.30 |
| 8.13E+00 | 3.62E−01 | 4.46 |
| 1.63E+01 | 5.12E−01 | 3.15 |
| 2.13E+01 | 5.87E−01 | 2.75 |
| 6.50E+01 | 1.02E−00 | 1.58 |
| 1.30E+02 | 1.45E−00 | 1.11 |

The inkjet technique has revealed that the accuracy at which one yeast containing an IPC nucleic acid in a predetermined copy number of 1, i.e., 1 copy of IPC nucleic acid is dispensed in a well is ±0.1281 copy. The accuracy at which one or more copies are dispensed in a well can be determined by accumulating data of this accuracy.

Using the above-described results, the expanded uncertainty obtained as an index for a dispersion of measurement is memorized as data for the device, whereby the index of uncertainty can be used as a criterion of evaluation for the reliability of the measurement result of each well. Using the above-described criterion of evaluation for the reliability allows performing the performance evaluation of an analysis test with high accuracy.

(4) Allocation of Value of Uncertainty to Each Site of Filling

The above-described calculated uncertainty (or coefficient of variation) was allocated as a value to each well.

Using the above-mentioned results, the average copy number of nucleic acids in a low-concentration nucleic acid specimen series and the uncertainty and coefficient of variation of the copy number were calculated, enabling a value to be allocated to each well.

### Example 2

(Purpose)

The inhibition that the addition of an IPC nucleic acid caused a nucleic acid amplification reaction was evaluated.

(Method)

As an IPC nucleic acid, 6000 used in Example 1 was used, and, as a test nucleic acid specimen, a human genomic DNA was used. A target nucleic acid was detected using EGFR-F (AGGTGACCCTTGTCTCTGTG: SEQ ID NO: 1) and EGFR-R (CCTCAAGAGAGCTTGGTTGG: SEQ ID NO: 2) as primers that amplify the EGFR gene. An IPC nucleic acid was detected using 600G-F (TCGAAGGGTGATTGGATCGG: SEQ ID NO: 4) and 600G-R (TGGCTAGCTAAGTGCCATCC: SEQ ID NO: 5) as primers that amplify 600G.

Into the wells as nucleic acid receiving parts, 600Gs as IPC nucleic acids were dispensed in 0, 10, 25, 50, 100, 500, 1000, 5000, and 10000 copies respectively, a total of 9 levels, which were each for 8 wells. A method for dispensing 100 copies or less was performed by the same procedures as in Example 1, and 500 copies or more were dispensed by pipetting. The copy number of 500 copies or more was estimated from a value of a measurement made preliminarily by digital PCR.

Next, into each of the wells into which IPC nucleic acids had been dispensed, human genomic DNAs were dispensed in 100, 500, 1000, and 5000 copies respectively, i.e., 4 levels, which were each for 2 wells. A method for dispensing 100 copies or less was performed by the same procedures as in Example 1, and 500 copies or more were dispensed by pipetting. The copy number of 500 copies or more was estimated from a value of a measurement made preliminarily by digital PCR. Then, in the wells, the IPC nucleic acids and the test nucleic acid specimens were used to perform an amplification reaction by PCR in the same well. The composition of the reaction liquid is tabulated in Table 3.

TABLE 3

| | Concen- | |
|---|---|---|
| | tration | |
| Reagent | (μM) | μL |
| TagMan ® Universal PCR Master Mix | | 12 |
| Forward Primer EGFR | 10 | 1.2 |
| Reverse Primer EGFR | 10 | 1.2 |
| TaqMan ® Probe (FAM) EGFR | 20 | 0.24 |
| Forward Primer 600 G | 10 | 1.2 |
| Reverse Primer 600 G | 10 | 1.2 |
| TaqMan ® Probe (HEX) 600 G | 20 | 0.24 |
| NFW | | 0.72 |
| DNA (IPC: 600 G) | | 4 |
| DNA (Sample: Human gDNA) | | 2 |
| reaction mix total volume | | 24 |

The amplification reaction was performed by PCR, using a QuantStudio™ 12K Flex real-time PCR system (Thermo Fisher Scientific Inc.). The reaction conditions for the PCR were as follows: incubation at 50° C. for 2 minutes was followed by incubation at 95° C. for 10 minutes, and followed by performing, 50 times, a temperature cycle composed of two steps, one at 95° C. for 30 seconds, and the other at 61° C. for 1 minute. The Ct value of each well was outputted, and graphed.

(Results)

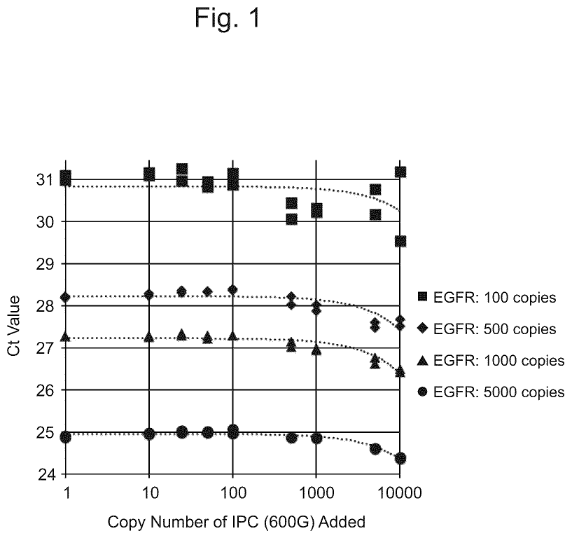

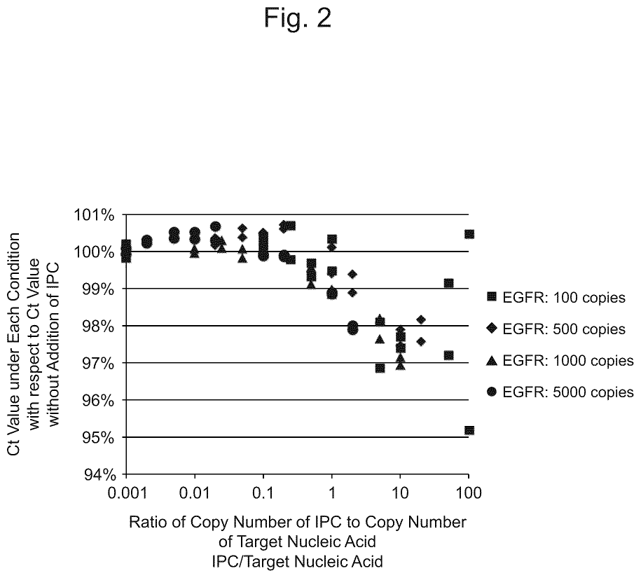

The results are graphed in FIG. 1 and FIG. 2.

FIG. 1 has verified that the Ct value of the EGFR gene as a target nucleic acid, not depending on the copy number, was decreased under all conditions as the copy number of IPC nucleic acids increased. In cases where 100 copies of the EGFR genes were used, an increase in the dispersion was recognized as the copy number of IPC nucleic acids increased.

FIG. 2 illustrates the same data from a different viewpoint. Specifically, the ordinate is the ratio (%) of the Ct value under each condition when Ct value with no IPC nucleic acid added, i.e., the Ct value of the EGFR gene with 0 copy of 600G was 100%, and the abscissa was the ratio of the copy number of 600Gs to the copy number of the EGFR genes.

The result verified was that, when the ratio of IPC nucleic acids/target nucleic acids was more than 1, the Ct value was decreased, compared with the addition of no IPC nucleic acid.

### Example 3

(Purpose)

The result that the plateau position in PCR was lowered by the addition of IPC nucleic acids was evaluated.

(Method)

In the same manner as in Example 2, 600G was used as an IPC nucleic acid, and a human genomic DNA was used as a test nucleic acid specimen. To detect a target nucleic acid, the same primers, EGFR-F and EGFR-R, as in Example 2 were used. To detect an IPC nucleic acid, the same 600G-F and 600G-R as in Example 2 were used. Furthermore, as a probe for detecting the target nucleic acid, an EGFR-probe (AGCTTGTGGAGCCTCTTACACCCAGT: SEQ ID NO: 3) was used, and as a probe for detecting an IPC nucleic acid, a 600G-probe (TGCATTCTGGCTTCGATTGTCCCTAC: SEQ ID NO: 6) was used.

Into the wells as nucleic acid receiving parts, IPC nucleic acids and test nucleic acid specimens were dispensed by the same arrangement as in Example 2. Then, the IPC nucleic acids and the test nucleic acid specimens were used to perform an amplification reaction by PCR in the same well. The composition of the reaction liquid and the reaction conditions were as in Example 2.

(Results)

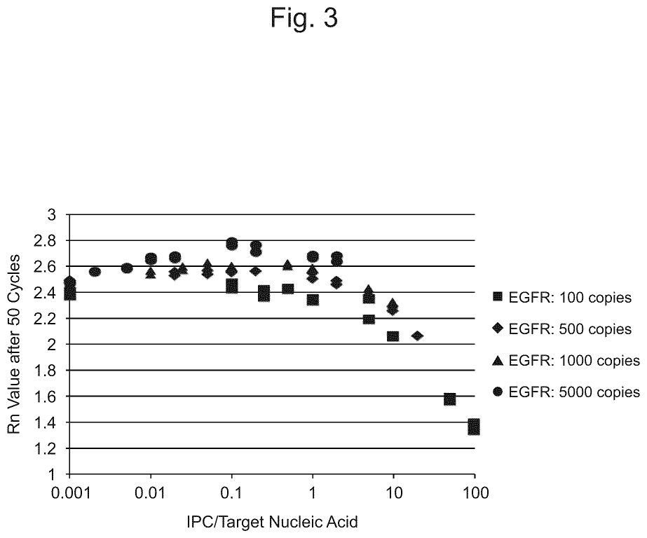

FIG. 3 graphs the Rn value outputted in each well after the 50 cycles. As above-described, the Rn value represents the intensity of fluorescent signal normalized by dividing the fluorescence emission intensity of a reporter pigment of a probe to be used for qPCR by the fluorescence emission intensity of a passive reference pigment. In FIG. 3, the ordinate represents the Rn value after the 50 cycles, and the abscissa represents the ratio of the copy number of 600Gs as IPC nucleic acids to the copy number of EGFR genes as target nucleic acids.

The result of FIG. 3 has revealed that, if the value (ratio) of IPC nucleic acids/target nucleic acids is 3 or less, the Rn value after the 50 cycles, i.e., the position of plateau was equal to or higher than the value (approximately 2.4) at the ratio of 0.001 (corresponding to the target nucleic acids at 100% in FIG. 3) with any copy number. On the other hand, the result verified that, if the ratio was more than 3, the Rn value was markedly decreased.

### Example 4

(Purpose)

The result that the plateau position in PCR was lowered by the addition of IPC nucleic acids was evaluated by electrophoresis.

(Method)

In the present Example, 600G and a human genomic DNA were used as an IPC nucleic acid and a test nucleic acid specimen respectively in the same manner as in Examples 2 and 3. To detect a target nucleic acid, the same primers, EGFR-F and EGFR-R, as in Example 2 were used.

Into the wells as nucleic acid receiving parts, IPC nucleic acids and test nucleic acid specimens were dispensed by the same arrangement as in Example 2. The levels of the IPC nucleic acids were 0, 10, 25, 50, 100, 500, 1000, 5000, and 10000 copies, a total of 9 levels, in the same manner as in Examples 2 and 3. The dispensing method was in accordance with the method in Example 2. The test nucleic acid specimens were added to all of the wells in 100 copies each. Then, the IPC nucleic acids and the test nucleic acid specimens were used to perform an amplification reaction by PCR in the same well. The composition of the reaction liquid and the reaction conditions were as in Example 2.

(Results)

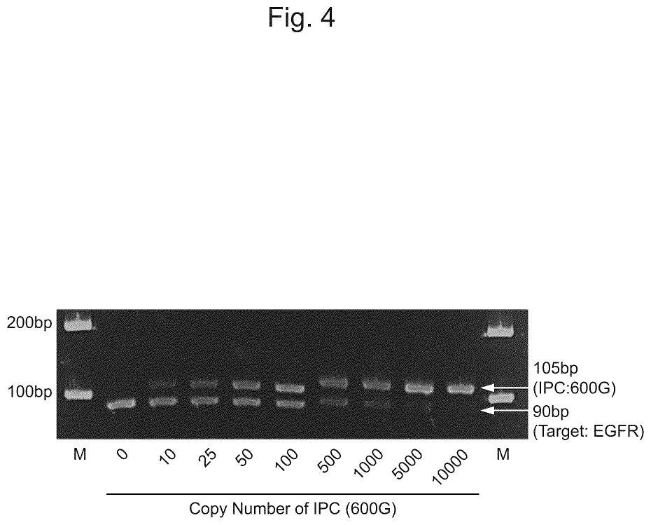

FIG. 4 is an electrophoretic image illustrating how an amplification product was electrophoresed with a 4% Agarose gel. As the result of PCR performed with the copy number fixed to 100 copies for the EGFR gene as a target nucleic acid, the brightness of the electrophoretic band (arrows) of the EGFR gene was decreased as the copy number of 600Gs as IPC nucleic acids was increased, as recognized. The result has revealed that, under the condition where the IPC nucleic acids were 100 times more than the target nucleic acids, i.e., EGFR:600G=100 copies:10000 copies, no electrophoretic band of the EGFR gene was recognized, representing a false-negative state.

### Example 5

(Purpose)

How the linearity of a measurement value was affected by the inhibition that the addition of IPC nucleic acids caused a nucleic acid amplification reaction was evaluated.

(Method)

As an IPC nucleic acid, 600G used in Example 1 was used, and as a test nucleic acid specimen, the novel coronavirus detection sequence N2 was used. The N2 is part of the nucleocapsid protein coding region of the novel coronavirus. The target nucleic acid was detected using N2-F (TTACAAACATTGGCCGCAAA: SEQ ID NO: 7) and N2-R (GCGCGACATTCCGAAGAA: SEQ ID NO: 8) as amplification primers for the N2 region of the novel coronavirus. The IPC nucleic acid was detected using the 600G-F (SEQ ID NO: 4) and 600G-R (SEQ ID NO: 5) that were used in Example 2.

Into the wells as nucleic acid receiving parts, 600Gs as IPC nucleic acids were dispensed in 0, 10, 100, 1000, 10000, and 100000 copies respectively, a total of 6 levels, which were each for 16 wells. A method for dispensing 100 copies or less was performed by the same procedures as in Example 1, and 1000 copies or more were dispensed by pipetting. The copy number of 500 copies or more was estimated from a value of a measurement made preliminarily by digital PCR.

Next, nucleic acids containing the N2 sequence of the novel coronavirus were prepared by the same procedures as in Example 1, and dispensed into each well. Into each of the wells, the nucleic acids were dispensed in 0, 5, 50, 500, 5000, and 50000 copies respectively. i.e., 6 levels, which were each for 12 wells. A method for dispensing 50 copies or less was performed by the same procedures as in Example 1, and 500 copies or more were dispensed by pipetting. The copy number of 500 copies or more was estimated from a value of a measurement made preliminarily by digital PCR. Then, in the wells, the IPC nucleic acids and the test nucleic acid specimens were used to perform an amplification reaction by qPCR in the same well. The composition of the reaction liquid is tabulated in Table 4.

TABLE 4

| Component | μL/well |
|---|---|
| Luna ® Universal Probe One-Step Reaction Mix | 10.0 |
| Luna ® WarmStart ® RT-Enzyme Mix | 1.0 |
| N2 Forward primer (20 μM) | 0.4 |
| N2 Reverse primer (20 μM) | 0.4 |
| N2 FAM-BHQ1 probe (10 μM) | 0.4 |
| DNA600G Forward primer (20 μM) | 0.4 |
| DNA600G Reverse primer (20 μM) | 0.4 |
| DNA600G HEX-BHQ1 probe (10 μM) | 0.4 |
| NFW | 0.6 |
| DNA600G | 2.0 |
| Template | 4.0 |
| Total | 20.0 |

The amplification reaction was performed by PCR, using LightCycler (registered trademark) 480 System II 96-well (Roche Diagnostics K.K.). The reaction conditions for the PCR were as follows: incubation at 55° C. for 10 minutes was followed by incubation at 95° C. for 1 minute, and followed by performing, 50 times, a temperature cycle composed of two steps, one at 95° C. for 10 seconds, and the other at 60° C. for 1 minute. The Ct value of each well was outputted and graphed.

(Results)

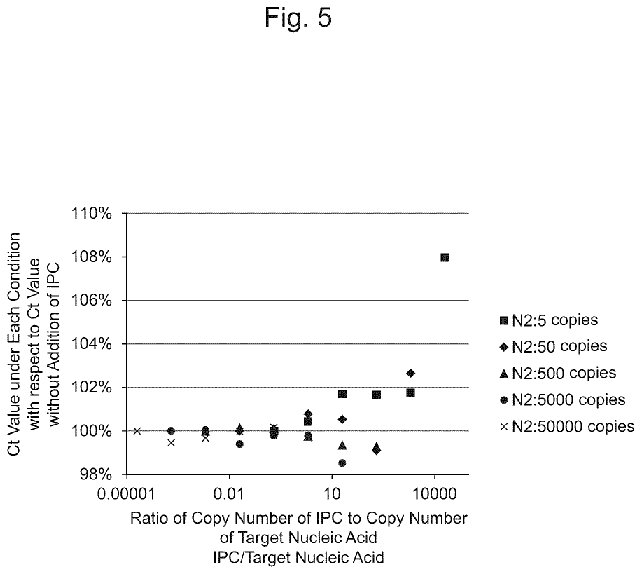

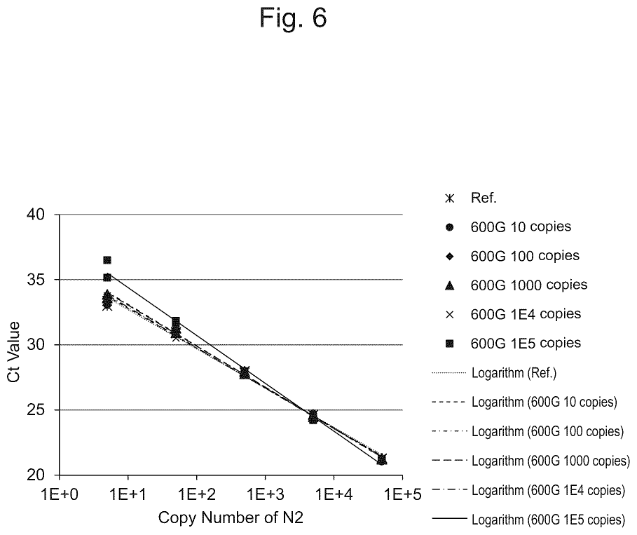

The results are graphed in FIG. 5 and FIG. 6.

According to FIG. 5, the Ct value of the novel coronavirus N2 sequence as a target nucleic acid was increased as the copy number of the IPC nucleic acids added was increased. However, as illustrated in FIG. 2, Example 2 resulted oppositely in a decrease with an increase in the copy number of the IPC nucleic acids. These results suggest that the Ct value is increased or decreased with an increase in the copy number of the IPC nucleic acids, depending on the kind of the target nucleic acid. On the other hand, the Ct value in FIG. 5 exhibited an increase trend with a low copy number (approximately 50 copies) of target nucleic acids, and a decrease trend in the same manner as in Example 2 with a high-copy (500 copies or more). As a cause of this, a problem of inhibition caused to amplification by competition in the PCR reaction was assumed for the low copy, and a problem with the equipment that detected the fluorescence of HEX as the fluorescence of FAM owing to the overlap of the fluorescence peaks of FAM and HEX was assumed for the high copy. Either case has revealed that the Ct value caused unintended vanations as the copy number of the IPC nucleic acids increased. FIG. 5 has verified that the variations in the Ct values were reduced to +2% or less in cases where the ratio of the copy number of the IPC nucleic acids to the estimated copy number of the target nucleic acids (IPCs/target nucleic acids) was 200 in the detection of the N2 region of the novel coronavirus.

FIG. 6 illustrates the same data from a different viewpoint. Specifically, the data represent a calibration curve exhibiting the relationship between two values: the ordinate representing the Ct values of the N2 sequence of the novel coronavirus:and the abscissa representing the copy number of the N2 sequences. With this calibration curve, the copy number of target nucleic acids (here, the N2 sequences) the concentration of which is unknown can be estimated from the Ct value. However, in cases where the linearity is impaired by the addition of IPCs, the purpose is not achieved. In FIG. 6, in cases where 600Gs as IPCs were added, a linearity was recognized, up to 10000 copies (×), in the same manner as in cases where 600G was not added (*). However, in the low copy number region of the N2, the value for 100000 copies (▪) is out into a higher side than the straight line for the no addition (*), and the linearity is lost, as illustrated. This result has revealed that, in a nucleic acid detection system using a target nucleic acid detection device according to the present invention, the linearity for 5 to 50000 copies was maintained even in cases where 10000 copies of IPCs were added.

### RELATED ART DOCUMENTS

### Patent Documents

[Patent Document 1] Japanese Unexamined Patent Application Publication No. 2014-33658
[Patent Document 2] Japanese Unexamined Patent Application Publication No. 2015-195735
[Patent Document 3] Japanese Unexamined Patent Application Publication No. 2008-245612
[Patent Document 4] Japanese Patent No. 4805158

### Non-Patent Documents

[Non-Patent Document 1] Okuda, M., 2008, “Syokubutu Boheki”, 62(12):20-22.
[Non-Patent Document 2] Zachar, V., et al., 1993, Nucleic Acid Research, 21(8):2017-2018.
[Non-Patent Document 3] Gilliland, G., et al., 1990, Proc. Natl. Acad. Sci., 87:2725-2729

All the publications, patents, and patent applications cited herein are incorporated herein by reference in their entirety.

1: A target nucleic acid detection device, comprising:
a base material, and
a nucleic acid receiving part,
the nucleic acid receiving part containing a predetermined copy number of internal positive control (IPC) nucleic acids either on a surface of or in an internal space of or both on the surface of and in the internal space of the nucleic acid receiving part, the IPC nucleic acid having a specific base sequence, and
the predetermined copy number being a copy number according to which a ratio of the copy number of the IPC nucleic acids to an estimated copy number of target nucleic acids is 200 or less.

2: The target nucleic acid detection device according to claim 1, wherein the copy number of the IPC nucleic acids is 10000 copies or less.

3: The target nucleic acid detection device according to claim 1, comprising two or more of the nucleic acid receiving parts.

4: The target nucleic acid detection device according to claim 3, wherein the copy number of the IPC nucleic acids contained in each nucleic acid receiving part is the same between the nucleic acid receiving parts.

5: The target nucleic acid detection device according to claim 3, wherein at least one of the nucleic acid receiving parts contains a positive control (PC) nucleic acid in place of the target nucleic acid.

6: The target nucleic acid detection device according to claim 5, comprising a plurality of the nucleic acid receiving parts, each of which contains PC nucleic acid specimens in different copy numbers.

7: The device according to claim 5, further comprising a PC nucleic acid receiving part containing a PC nucleic acid alone.

8: The target nucleic acid detection device according to claim 7, wherein a copy number of the PC nucleic acids in the PC nucleic acid receiving part is the same as a copy number of the PC nucleic acids in at least one of the nucleic acid receiving parts.

9: A target nucleic acid detection kit, comprising:
the target nucleic acid detection device according to claim 1, and
an IPC amplification primer.

10: A method for producing a target nucleic acid detection device, comprising:
dispensing a predetermined number of cells into one or more nucleic acid receiving parts on a base material, the cell containing a predetermined copy number of IPC nucleic acids having a specific base sequence; and
extracting a nucleic acid from the cell.

11: The production method according to claim 10, wherein the nucleic acid in the cell is labeled before the IPC dispensing.

12: The production method according to claim 10, wherein the copy number of the IPC nucleic acids is 10000 copies or less.

13: The production method according to claim 10, wherein, in cases where the cells are dispensed into a plurality of the nucleic acid receiving parts in the IPC dispensing step, the cells are dispensed in such a manner that the copy number of the IPC nucleic acids contained in each nucleic acid receiving part is the same between the nucleic acid receiving parts.

14: The production method according to claim 10, further comprising:
dispensing, into any one or more of the nucleic acid receiving parts, a predetermined number of cells containing a predetermined copy number of PC nucleic acids in cases where the cells are dispensed into a plurality of the nucleic acid receiving parts in the IPC dispensing.

15: The production method according to claim 13, wherein the nucleic acid in the cell is labeled before the PC dispensing.

16: The production method according to claim 14, wherein a ratio of the copy number of the IPC nucleic acids to the copy number of the PC nucleic acids is 200 or less per nucleic acid receiving part.

17: The production method according to claim 10, wherein the dispensing is performed by an inkjet technique.


SEQUENCE LISTING (8 sequences)

SEQ ID NO: 1 - DNA, 20 bp, synthetic construct
  misc_feature 1..20: note = primer (EGFR-F)
  source 1..20: mol_type = other DNA
aggtgacccttgtctctgtg

SEQ ID NO: 2 - DNA, 20 bp, synthetic construct
  misc_feature 1..20: note = primer (EGFR-R)
  source 1..20: mol_type = other DNA
cctcaagagagcttggttgg

SEQ ID NO: 3 - DNA, 26 bp, synthetic construct
  misc_feature 1..26: note = probe
  source 1..26: mol_type = other DNA
agcttgtggagcctcttacacccagt

SEQ ID NO: 4 - DNA, 20 bp, synthetic construct
  misc_feature 1..20: note = primer (600G-F)
  source 1..20: mol_type = other DNA
tcgaagggtgattggatcgg

SEQ ID NO: 5 - DNA, 20 bp, synthetic construct
  misc_feature 1..20: note = primer (600G-R)
  source 1..20: mol_type = other DNA
tggctagctaagtgccatcc

SEQ ID NO: 6 - DNA, 26 bp, synthetic construct
  misc_feature 1..26: note = probe
  source 1..26: mol_type = other DNA
tgcattctggcttcgattgtccctac

SEQ ID NO: 7 - DNA, 20 bp, synthetic construct
  misc_feature 1..20: note = primer (N2-F)
  source 1..20: mol_type = other DNA
ttacaaacattggccgcaaa

SEQ ID NO: 8 - DNA, 18 bp, synthetic construct
  misc_feature 1..18: note = primer (N2-R)
  source 1..18: mol_type = other DNA
gcgcgacattccgaagaa